(19) United States
(12) Patent Application Publication
(10) Pub. No.: US 2004/0156839 A1
(43) Pub. Date: Aug. 12, 2004

(54) Antisense peptides to human fc receptors and their uses

(76) Inventors: Katherine Lucy Dry, Cambridge (GB); Joseph Michael Sheridan, Herfordshire (GB); John Graham Raynes, St. Albans (GB)

Correspondence Address: FINNEGAN, HENDERSON, FARABOW, GARRETT & DUNNER, LLP, 1300 I STREET, NW, WASHINGTON, DC, 20005, US

(21) Appl. No.: 10/399,162

(22) PCT Filed: Oct. 10, 2001
(86) PCT No.: PCT/GB01/04483

(30) Foreign Application Priority Data
Oct. 14, 2000 (GB) 0025251.0

(51) Int. Cl. A61K 39/395; C12N 5/06; C07K 16/44

(52) U.S. Cl. 424/130.1; 435/327; 530/387.3

(57) ABSTRACT

This invention describes the design and therapeutic use of antisense peptides for immunomodulation. Specifically applications include blocking the interaction of immunoglobulin G (IgG) with Fcγ receptors (FCγR) and blocking the interaction of immunoglobulin E (IgE) with FcεRI or IgA with FcαRI. This invention also describes the application of antisense peptides to target vaccines to FcγR to induce or suppress immunity, and also describes the application of antisense peptide specific for FcγR or FcαRI to direct cytotoxic effector cells to target cells or pathogens.

### SUMMARY

This invention describes the design and therapeutic use of antisense peptides for immunomodulation. Specifically applications include blocking the interaction of immunoglobulin G (IgG) with Fcγ receptors (FcγR) and blocking the interaction of immunoglobulin E (IgE) with FcεRI or IgA with FcαRI. This invention also describes the application of antisense peptides to target vaccines to FcγR to induce or suppress immunity, and also describes the application of antisense peptide specific for FcγR or FcαRI to direct cytotoxic effector cells to target cells or pathogens.

### BACKGROUND OF THE INVENTION

Fc Receptors (FcR)

The Fcγ receptors (FcγR), FcεRI and FcαRI are homologous type I membrane proteins that bind the Fc portions of IgG, IgE and IgA respectively. In humans the Fc γR are expressed on most immunocompetent cells and FcεRI is expressed on mast cells, basophils, monocytes, eosinophils, platelets, Langerhans cells, and dendritic cells. The FcγR are split into three main groups FcγRI (CD64), FcγRII (CD32) and FcγRIII (CD16) (Gessner et al. 1998; Kinet 1999). FcαRI (CD89) is expressed on macrophages/monocytes, eosinophils and granulocytes (Wines et al. 1999). The FcγR, FcεRI and FcαRI have extracellular immunoglobulin superfamily (IgSF) domains with which they bind Fc. FcεRI, FcαRI and the low affinity IgG receptors, FcγRII and FcγRIII, all have two IgSF extracellular (EC) domains (EC1 and EC2) whereas FcγRI has an additional unique third IgSF domain (EC3). EC1 and EC2 domains of FcγR and FcεRI share approximately 40% sequence identity. FcαRI is more distantly related to the other members of the FcR family with approximately 20% sequence homology. Mutagenic and chimeric studies of FcγRII, FcγRIII and FcεRI indicated that the membrane proximal domain (EC2)is the Fc binding site. Crystal structures of FcγRIII complexed with the Fc portion of IgG1 and FcεRI complexed with the Fc portion of IgE confirms the binding of Fc to the EC2 domains, and shows additional binding of Fc to the EC1-EC2 linker (Garman et al. 2000; Sondermann et al. 2000). Binding of IgG Fc to the high affinity FcγR, FcγRI, is mediated by EC2 in conjunction with a third IgSF domain (EC3) although the exact role of EC3 remains unclear. In contrast, the IgA binding site of FcαRI is in the EC1 domain (Wines et al. 1999).

The FcγR have a central role in the initiation of cellular responses against pathogens and soluble antigens, mediating the clearance of immune complexes by their internalization and breakdown. The cross-linking of FcγR by IgG immune complexes triggers the immune response. The resultant cellular responses are complex and range from endocytosis and antibody-dependent cellular cytotoxicity (ADCC), to the secretion of inflammatory mediators, enhanced MHC class II presentation of T cell epitopes and the regulation of antibody production. Receptors either act through a gamma chain (FcγRI, FcεRI) or an immunoreceptor tyrosine-based activating motif (ITAM, FcγRIIa) or immunoreceptor tyrosine-based inhibition motif (ITIM, FcγRIIb).

FcεRI binds IgE with high affinity. Antigen (allergen)-induced Igh cross-linking results in redistribution of FcεRI on the cell surface leading to activation and degranulation of mast cells and basophils and a release of mediators of the allergic response that are associated with a type I IgE-dependent allergic reaction.

Therapeutic Applications

Use of Antisense Peptides to Block the Interaction of FcγR with IgG

Antisense peptide-based inhibitors of the interaction of FcγR with IgG could therapeutically down regulate the immune system. Conditions where inhibitors of the interaction of FcγR with IgG would be therapeutic include: autoimmune diseases such as systemic lupus erythematosus (SLE), autoimmune glomerulonephritis and immune thrombocytopenic purpura (ITP); graft versus host rejections; FcγRIIb inhibition in tumour therapies (Rouard et al. 1997; Clynes et al. 1998; Clynes et al. 2000; Marino et al. 2000). Antisense peptide-based inhibitors of the interaction of FcγR with IgG would be designed to bind to the protein sequence motifs of FcγR involved in interaction with IgG.

B cell activation is regulated by FcγRII and FcγRIII in combination with other receptors (BCR) and B cell development is also partly regulated by interaction with Fc γRII and FcγRIII. Thus antibody production and control of B cell lineage development and activity may be modulated by antisense peptides that alter interaction of antigen/immunoglobulin complexes with ITIM (inhibitory, FcγRIIb) or ITAM (activating, FcγRIIa) containing receptors or γ-chain associated receptors (activating, FcγRIII).

Use of Antisense Peptide to Block the Interaction of FcεRI with IgE

Antisense peptide-based inhibitors of the interaction of IgE with FcεRI could be therapeutic in atopic disorders such as asthma, allergic rhinitis, urticaria, angiodema and atopic dermatitis. Clinical studies of allergic individuals using anti-IgE monoclonal antibody therapy has shown that this is an effective approach to allergic disease treatment (Chang 2000): Antisense peptide-based inhibitors of the interaction of FcεRI with IgE would be designed to bind to the protein sequence motifs of FcεRI involved in interaction with IgE.

Antisense Peptide Targeting of FcγR to Promote T Cell Activation or Anergy In Vivo.

An antisense peptide-FcγR ligand could be conjugated to a vaccine that contains strong or anergic T cell epitope/s for the purpose of modulating T cell activation. The use of native and modified T cell epitopes to induce or suppress immunity requires efficient uptake and processing by antigen-presenting cells (APC) in vivo. Normally, FcγR expressed on APC internalize antigen-antibody complexes and thus induce processing of antigens into peptides (T cell epitopes) which are subsequently presented by major histocompatibility complex (MHC) class II molecules to T cells. Enhanced presentation of antigenic and antagonistic peptides has been demonstrated by targeting the peptides to FcγRI (CD64) which is expressed constitutively by APC (reviewed in Guyre et al. 1997). The antisense peptide specific for the FcγR could be designed to bind in, or alternatively outside, the IgG binding site. An antisense peptide that binds to FcγR outside of the IgG binding site would not be blocked by circulating human IgG.

Use of Antisense Peptides Specific for FcγR or FcαRI to Direct Cytotoxic Effector Cells to Target Cells or Pathogens.

An antisense peptide specific for FcγR (e.g. FcγRI) or FcαRI could be used to direct cytotoxic effector cells (e.g. macrophages) against target cells of pathogens. The directed effector cells can be used to kill target cells by cell-mediated cytolysis. The target cell can be a cell, such as a cancer cell, whose elimination would be beneficial to the host. It has been demonstrated that target cell conjugation and lysis can be induced by bispecific antibodies specific for both a target cell epitope and an FcγR (Rouard et al. 1997). It has also been demonstrated that enhanced pathogen phagocytosis and killing can be induced by bispecific antibodies specific for both the pathogen and an FcγR or FcαRI (van Spriel et al. 1999). As an alternative, an antisense peptide specific for the appropriate FcγR or FcαRI could be conjugated to an agent that is specific for a target cell epitope or pathogen. The agent that is specific for a target cell or pathogen epitope could also be an antisense peptide. The antisense peptide specific for FcγR or FcαRI could be designed to bind in, or alternatively outside, the IgG or IgA binding sites respectively. An antisense peptide that binds to FcγR or FcαRI outside of the IgG or IgA binding sites would not be blocked by circulating human IgG or IgA.

### DESCRIPTION OF THE INVENTION

The present invention refers to peptides whose sequence has been defined in such a way that they are antisense in sequence to the target protein. Such peptides have been shown to bind to their target protein. This approach has been applied to the immunoglobulin binding regions of the following receptors:

FcγRI

FcγRIIa

FcγRIb

FcγRIII

FcεRI

FcαRI

Recently the structures of several Fcγ receptors complexed with their ligands have been described (Garman et al., 2000, Maxwell et al., 1999; Sondermann et al., 1999; Sondermann et al., 2000). These show similar structure and mutational studies have implicated a number of regions the B/C, C′/E, F/G and CC′ loops of EC2 and the EC1-EC2 linker region as important for binding to the IgG molecule. The EC1 domain of FcαRI has been implicated as important for binding IgA (Wines et al. 1999).

Antisense peptides were designed to the binding regions of FcγRI, FcγRIIa, FcγRIIb, FcγRIII, FcεRI and FcαRI on the basis that a peptide designed from the antisense strand of a receptor will bind to its corresponding sense peptide. The peptides of the invention are of an integral number of amino acids in the range of 5 to 20 amino acid.

### EXAMPLE 1

Describes the design of antisense peptides to the loop regions of FcγRI and their use as inhibitors of FcγRI activity.

Peptide Design

Peptides of 6 amino acids in length were designed to be antisense in sequence to the corresponding loop region of FcγRI. Peptides could, however, be any length ranging from 5 up to 20 amino acids in length.

Key loop region BC(128-133)*

Amino acid sequence:

KDKLVY

DNA sequence (418-436):

5′aag gat aag ctg gtg tac

Antisense DNA sequence:

5′gta cac cag ctt atc ctt

Antisense peptide sequence 1 (SEQ ID NO. 1)

VHQLIL

Key loop region C′E (146-151)*

Amino acid sequence:

FFHWNS

DNA sequence (472-490):

5′ ttt ttc cac tgg aat tct

Antisense DNA sequence:

5′ aga att cca gtgcgaa aaa

Antisense peptide sequence 2 (SEQ ID NO.2)

RIPVEK

Key loop region FG (172-177)*

Amino acid sequence:

GKHRYT

DNA sequence (550-568):

5′ gga aag cat cgc tac aca

Antisense DNA sequence:

5′ tgt gta gcg atg ctt tcc

Antisense peptide sequence 3 (SEQ ID NO.3):

CVAMLS

*Numbers in brackets refer to the amino acid position in the full-length protein.

The 3 peptides were synthesized using standard techniques.

Two separate assays were used to assess the binding and biological activity of these peptides.

1. Rosetting Assay

Rosetting is a measure of the ability of sheep red blood cells (SBRCs) to attach to the surface of Cos-7 cells. Binding could be via the FcγRI or other cell surface proteins.

Thus the inhibition of rosetting by antisense peptides designed to FcγRI shows inhibition of binding only and not the ability of a peptide to block phagocytosis that is triggered by a specific interaction of IaG with its receptor.

2. Phagrocytosis/Rosetting Assay for Fc Receptor-Peptide Interaction

The phagocytosis/rosetting assay described can be used to assess the ability of antisense peptides to specifically block the interaction of FcγRI expressed on the surface of transfected Cos 7 cells with IgG expressed on the surface of SRBCs. Following binding SRBCs are opsonized and phagocytosed by the Cos 7 cells only through binding to FcγRI. Any inhibition of phagocytosis by an antisense peptide must therefore be via a specific interaction of the antisense peptide with its receptor.

Both phagocytosis and rosetting require the expression of FcγRI on the surface of Cos 7 cells as described below.

Transfection of Cos 7 Cells

Cos-7 cells were maintained at 10⁵cells/ml. For transfection cells were seeded at 3-4×10⁵ cells/ml in growth medium and incubated overnight in 3 ml/60 mm petri dishes. Cells were transfected using the DEAE-dextran method with 10 ug of a fusion construct FcγRI-II DNA (comprising the extracellular domain of RI and the intracellular ITAM containing domain of RII) and allowed to recover at 37° C. overnight. Cells were then detached from the plates, resuspended at 10⁵ cells/ml and plated at 1 ml/well on coverslips in 24 well plates. The cells were left to re-adhere overnight.

Preparation of Sheep Red Blood Cells (SRBCs)

In order for SRBCs to adhere to and subsequently be phagocytosed by FcγRI expressing Cos 7 cells they require surface-bound IgG1 (opsonization). SRBCs were resuspended at 10⁵/ml in PBS±3 ul/ml of rabbit (IgG1 fraction) anti—SRBC stroma antibody (SIGMA). They were then rolled for 60 minutes at 4° C.

After 60 minutes they were washed three times with PBS and resuspended at 4×10⁶ and 20×10⁶ in PBS. Unopsonized (i.e. no surface bound IgG1) were used as controls. Unopsonized cells will not adhere to FcγRI expressing Cos 7 cells.

Preparation of Peptides

Peptides were dissolved in dH₂O and made up to a concentration of 20 ug/ml and 200 ug/ml in PBS.

Rosetting Assay Method

The medium was removed from each plate of cells, and 0.25 ml of opsonised or unopsonised, washed SRBCs were added to the wells of cells, also 250 μl/well of peptides 1-3 were added to their respective wells. Plates were incubated for 2½ hours at 37° C, and were washed 3 times with warm PBS. Cells were fixed for 5 minutes. Positive cells were identified by light microscopy. Rosetted cells (i.e. Cos7 cells with more than one SRBC attached to their surface) were scored ++ (approximately 75% of cells totally surrounded by SRBCs),+(approximately 40% of cells with many SRBCs),±(approximately 10% of cells with few SRBCs attached) and—(no attachment of SRBCs).

Phagocytosis Assay Method

The medium was removed from each plate of cells, and 0.25 ml of opsonised or unopsonised, washed SRBCs were added to the wells of cells, also 250 μl/well of peptides 1-3 were added to their respective wells. Plates were incubated for 2½ hours at 37° C., and were washed 3 times with warm PBS. To assess phagocytosis any surface bound SRBCs were first lysed with hypotonic shock buffer (150 ml dH20, 1 ml PBS and 50 μl of conc HCl for exactly 2 mins). Cells were then fixed for 5 mins and stained for 15 minutes at room temperature with o-dianisidine stain:

“Stain and go”: 8.1 ml of 0.2M NaH₂PO₄ [1.2 g/50 ml], 1.9 ml of 0.2M Na₂HPO₄ [1.42 g/50 ml], 12 mls HANKS buffer (SIGMA), 1 ml o-dianisidine HCL (at 1.25 mg/ml H₂O, SIGMA) and 15 μl of H₂O₂ (30%). The cell-coated coverslips were removed and mounted on slides. Positive cells i.e. Cos 7 cells containing SRBCs were identified by light microscopy. Cells were counted within several randomly chosen fields of view and the number of Cos 7 cells containing phagocytosed SRBCs expressed as a percentage of the total number of cells. The number of SRBCs was counted and expressed as percentage positive cells or Phagocytic Index (the number of phagocytosed SRBCs/100 Cos 7 cells).

The peptides listed in example 1 (SEQ ID NOs 1-3) have been shown to inhibit both rosetting and phagocytosis carried out as described.

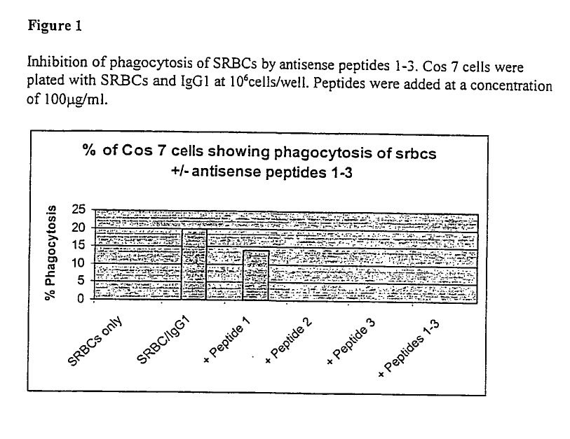

FIG. 1 shows an example of the inhibition of phagocytosis by antisense peptides 1-3 at a concentration of 100 μg/ml. A similar effect was observed with antisense peptides 1-3 at a concentration of 10 μg/ml.

Given the high degree of sequence similarity between the Fc receptors as illustrated in Sondermann et al., 2000, it is predicted that antisense peptides designed to the binding regions of FcγRIIa, FcγRIIb, FcγRIII, FcεRI and FcαR would show biological activity in similar or related bioassays. Antisense peptides have been designed to these regions and are listed in the Sequence Listing (SEQ ID NOs 4-17). The C′E loop of FcγRIIa has a polymorphism which has been shown to be important for IgG2 binding (H167) and IgG1 binding (R167) (Clark et al., 1989). Antisense peptides have been designed to both forms of the protein (SEQ ID NOs 5 and 6 respectively).

NB where the antisense peptide sequence contains a stop codon this has been replaced with a glycine residue.

References

Chang, T. W. (2000). The pharmacological basis of anti-IgE therapy. Nat Biotechnol 18(2): 157-62.

Clark, M. R., Clarkson, S. B., Ory, P. A., Stollman, N. & Goldstein, I. M. (1989). Molecular basis for a polymorphism involving Fc receptor II on human monocytes. J Immunol 143(5), 1731-4.

Clynes, R., C. Dumitru, et al. (1998). Uncoupling of immune complex formation and kidney damage in autoimmune glomerulonephritis. Science 279(5353): 1052-4.

Clynes, R. A., T. L. Towers, et al. (2000). “Inhibitory Fc receptors modulate in vivo cytoxicity against tumor targets [see comments].” Nat Med 6(4): 443-6.

Garman, S. C., B. A. Wurzburg, et al. (2000). Structure of the Fc fragment of human IgE bound to its high-affinity receptor Fc epsilonRI alpha. Nature 406(6793): 259-66.

Gessner, J. E., H. Heiken, et al. (1998). The IgG Fc receptor family. Ann Hematol 76(6): 231-48

Guyre, P. M., R. F. Graziano, et al. (1997). Increased potency of Fc-receptor-targeted antigens. Cancer Immunol Immunother 45(3-4): 146-8

Kinet, J. P. (1999). The high-affinity IgE receptor (Fc epsilon RI): from physiology to pathology. Annu Rev Immunol 17: 931-72.

Marino, M., M. Ruvo, et al. (2000). “Prevention of systemic lupus erythematosus in MRL/lpr mice by administration of an immunoglobulin-binding peptide.”Nat Biotechnol 18(7): 735-739.

Maxwell, K. F., Powell, M. S., Hulett, M. D., Barton, P. A., McKenzie, I. F., Garrett, T. P. & Hogarth, P. M. (1999). Crystal structure of the human leukocyte Fc receptor, Fc gammaRIIa. Nat Struct Biol 6(5), 437-42.

Rouard, H., S. Tamasdan, et al. (1997). Fc receptors as targets for immunotherapy. Int Rev Immunol 16(1-2): 147-85.

Sondermann, P., R. Huber, et al. (2000). The 3.2-A crystal structure of the human IgG1 Fc fragment-Fc gammaRIII complex. Nature 406(6793): 267-73.

Van Spriel, A. B., van den Herik-Oudijk, I. E. et al. (1999) Effective phagocytosis and killing of Candida albicans via targeting Fcgamma RI (CD64) or FcalphaRI (CD89) on neutrophils. J Infect Dis 179(3): 661-669.

Wines, B. D., Hulett, M. D., Jamieson, G. P., Trist, H. M., Spratt, J. M. & Hogarth, P. M. (1999). Identification of residues in the first domain of human Fcα receptor essential for interaction with IgA. J Immunol 162: 2146-2153.

5′gta cac cag ctt atc ctt (SEQ ID NO. 20)

Antisense peptide sequence 1 (SEQ ID NO.1)

VHQLIL

Please amend lines 1-17 on page 6 as follows:

Amino acid sequence:

FFHWNS (SEQ ID NO. 21)

DNA sequence (472-490):

5′ttt ttc cac tgg aat tct (SEQ ID NO 22)

Antisense DNA sequence:

5′aga att cca gtg gaa aaa (SEQ ID NO. 23)

Antisense peptide sequence 2 (SEQ ID NO.2)

RIPVEK

Key loop region FG (172-177)*

Amino acid sequence:

GKHRYT (SEQ ID NO. 24)

DNA sequence (550-568):

5′ gga aag cat cgc tac aca (SEQ ID NO. 25)

Antisense DNA sequence:

5′ tgt gta gcg atg ctt tcc (SEQ ID NO. 26)

1. An antisense peptide containing or consisting of an amino acid sequence that is antisense to a region of a protein selected from the group consisting of:
Human FcγRI
Human FcγRIIa
Human FcγRIIb
Human FcγRIII
Human FcεRI
Human FcαRI

2. An antisense peptide containing or consisting of an amino acid sequence that is antisense to a binding region of a protein selected from the group consisting of:
Human FcγRI
Human FcγRIIa
Human FcγR-IIb
Human FcγRIII
Human FcεRI
Human FcαRI

3. An antisense peptide as listed in any of SEQ ID NOS 1-17.

4. Use of an FcγR antisense peptide to block the interaction of FcγR with IgG.

5. Use of an FcγR antisense peptide as therapeutic agent in treatment of: autoimmune disease such as systemic lupus erythematosus (SLE), autoimmune glomerulonephritis and immune thrombocytopenic purpura (ITP); graft versus host rejection; FcγRIb inhibition in tumour therapy.

6. Use of an FcεRI antisense peptide to block the interaction of FcεRI with IgE.

7. Use of an FcεRI antisense peptide as therapeutic agent in atopic disorders such as asthma, allergic rhinitis, urticaria, angiodema and atopic dermatitis.

8. Use of an FcαRI antisense peptide to block the interaction of FcαRI with IgA.

9. Use of an FcγR antisense peptide to target FcγR to improve antigen processing and presentation for T cell activation or anergy in vivo.

10. Use of an antisense peptide specific for FcγR to direct cytotoxic effector cells to target cells or pathogens.

11. Use of an antisense peptide specific for FcαR to direct cytotoxic effector cells to target cells or pathogens.

12. Regulation of cellular activation and development of B cells through ITIM and ITAM containing FcγRIIa and FcγRIIb.

13. Use of a multimeric form of an antisense peptide according to any of claims 1-3 for any of the uses of claims 4-11.

14. Use of a multimeric antisense peptide which consists of or contains a combination of 2 or more antisense peptides (not all the same) according to any of claims 1-3 for any of the uses of claims 4-11.

15. A multimeric antisense peptide consisting of or containing 2 or more (the same or different) antisense peptides according to any of claims 1-3.
